(19) United States
(12) Patent Application Publication
(10) Pub. No.: US 2026/0169008 A1
(43) Pub. Date: Jun. 18, 2026

(54) BIOMARKER FOR TYPE 1 DIABETES

(71) Applicant: THE TRUSTEES OF INDIANA UNIVERSITY, Bloomington, IN (US)

(72) Inventors: Carmella EVANS-MOLINA, Zionsville, IN (US); Farooq SYED, Indianapolis, IN (US)

(21) Appl. No.: 18/710,034

(22) PCT Filed: Dec. 2, 2022
(86) PCT No.: PCT/US2022/051685

§ 371 Date: May 14, 2024

Related U.S. Application Data
(60) Provisional application No. 63/285,765, filed on Dec. 3, 2021.

(51) Int. Cl. G01N 33/68; A61K 31/56; A61K 45/06

(52) U.S. Cl. CPC G01N 33/6893; A61K 31/56; A61K 45/06; G01N 33/6803; G01N 2333/99; G01N 2474/10; G01N 2800/042; G01N 2800/50

(57) ABSTRACT

In islets collected from female NOD mice at 10, 12, and 14 weeks of age, there was a time-restricted upregulation of proteins involved in the maintenance of β cell function and stress mitigation, followed by loss of expression of these protective proteins that heralded diabetes onset. Pathway analysis showed modulation of EIF2 signaling, the unfolded protein response, mTOR signaling, mitochondrial function, and oxidative phosphorylation during disease progression in NOD mice and in the acute adoptive transfer model, highlighting the importance of this core set of pathways in TID pathogenesis. In immunofluorescence validation studies, β cell expression of protein disulfide isomerase A1 (PDIA1) and 14-3-3b were found to be increased during disease progression in NOD islets, while PDIA1 plasma levels were increased in pre-diabetic NOD mice and in the serum of children with recent-onset TID compared to age and sex-matched non-diabetic controls.

### CROSS REFERENCE TO RELATED APPLICATION

This application claims the benefit of and priority to U.S. Provisional Patent Application No. 63/285,765, filed on Dec. 3, 2021, the disclosure of which is expressly incorporated herein by reference in its entirety.

### GOVERNMENT LICENSE RIGHTS

This invention was made with government support under DK093954, DK127308, DK127786, and DK104166 awarded by the National Institutes of Health. The government has certain rights in the invention.

### BACKGROUND

Type 1 diabetes (T1D) results from immune-mediated destruction of the insulin-producing β cells and manifests clinically after a threshold reduction in β cell mass and function. Data from clinical cohorts of autoantibody-positive individuals followed longitudinally suggest that β cell function is impaired very early during disease progression, often years before the onset of clinical disease. In parallel, histologic studies performed on pancreatic sections from organ donors with autoantibody positivity and TID demonstrate variable reductions in β cell mass before and at diabetes onset. Findings from ex vivo disease models and pancreatic sections from human organ donors with diabetes have linked changes in β cell mass and function with the activation of a variety of β cell stress pathways that are thought to both accelerate β cell death and increase β cell immunogenicity.

A better understanding of the scope and timing of activation of these cell-intrinsic stress pathways has the potential to inform therapeutic and biomarker strategies in humans. At present, longitudinal imaging of the β cell compartment and sampling of the pancreas in living individuals is not clinically feasible, highlighting the need to interrogate alternative model systems. One commonly used model is the non-obese diabetic (NOD) mouse, which has been used to study TID pathogenesis for over three decades. Islets in NOD mice show evidence of immune cell infiltration as early as four weeks of age, and approximately 60-80% of female NOD mice develop diabetes by 16-20 weeks of age. Consistent with patterns observed in humans, β cell function and mass decline during the pre-diabetes phase. In cross-sectional analyses, subsets of overlapping stress pathways have been identified in β cells from NOD mice and human islets from organ donors with diabetes.

The present disclosure is directed towards discerning information on the timing and scope of these responses as well as disease-related changes in islet β cell protein expression during TID development. One aspect of the present disclosure is directed towards applying unbiased proteomics approaches in preclinical models to identify key β cell pathways involved in the temporal evolution of TID. Utilizing this strategy, a common set of modulated pathways across several distinct mouse models of T1D was identified.

### SUMMARY

Type 1 diabetes is discovered clinically at a time when there is extensive loss of beta cell mass and function. Therapies to prevent type 1 diabetes are more successful when given prior to the onset of clinical disease. Accordingly, biomarkers are needed to help identify beta cell stress and impending type 1 diabetes to allow for the administration of therapeutic methodologies to prevent or delay the onset of clinical type 1 diabetes. Therapies to treat or slow the progression of type 1 diabetes include potential immunomodulatory therapies such as an anti-CD3 monoclonal antibody. However, a key question remains as to the ideal timing of such a therapy in individuals who are at risk of developing diabetes.

This disclosure is directed towards the use of a discovery-based SWATH proteomics approach to monitor longitudinal changes in islet protein expression during early and late disease progression in NOD mice to gain additional insight into the time course of molecular changes in the β cell during TID progression. Proteomic signatures in islets from diabetic NOD mice was compared with those observed in islets from NOD-SCID mice rendered acutely diabetic by the adoptive transfer of T cells from NOD-BDC2.5 mice. Finally, to gain insight into potentially protective pathways, proteomes generated from NOD mouse islets at the time of diabetes onset were compared to those from NOD mice that remained diabetes free through 48 weeks of age. To illustrate the utility of this approach in prioritizing β cell proteins as TID biomarkers, analysis was focused on protein disulfide isomerase A1 (PDIA1) as an example of a secreted protein that was found to be differentially expressed in NOD islets during diabetes progression. PDIA1 was selected as a target for the development of a high sensitivity electrochemiluminescence assay using MesoScale Discovery technology. Using this assay, increased β cell expression and plasma concentrations of PDIA1 was demonstrated in NOD mice during the evolution of T1D and in the serum of children with recent-onset T1D when compared to age and sex-matched pediatric controls.

As disclosed herein, experimental model systems are used to identify early biomarkers that are associated with and identify beta cell stress and impending type 1 diabetes. In accordance with one embodiment, an assay is provide for measuring PDIA1 levels in bodily fluids, such as blood or serum. This assay has been used to demonstrate that PDIA1 levels are increased in the mouse model of type 1 diabetes and the blood of persons with recent onset type 1 diabetes compared to healthy subjects (controls). Accordingly, in one embodiment elevated levels of PDIA1 are used as a diagnostic marker of beta cell stress and impending type 1 diabetes. In one embodiment PDIA1 is identified as a potential T1D associated biomarker in humans.

In accordance with one embodiment, a method of identifying and measuring biomarkers in the blood of persons with recent onset type 1 diabetes is used to identify early diagnostic markers of subjects at risk of developing type 1 diabetes.

In accordance with one embodiment of the present disclosure, the method comprises the steps of analyzing the proteome of islets collected from female NOD mice at three pre-diabetic time points and the time of diabetes onset; analyzing the proteome of islets from diabetic NOD mice and NOD-SCID mice that has been rendered acutely diabetic following the adoptive transfer of T cells from NOD.BDC2.5 mice; analyzing the islet proteomes of NOD mice that remained diabetes-free after 46-48 weeks of observation, identifying biomarkers whose expression is altered in each of the three analyzed proteomes, and using the biomarkers before administering a type 1 diabetes preventative therapeutic in humans.

In one embodiment, the overall concentration of proteins is measured in each islet proteome. In one embodiment, the concentration of proteins is measured relative to the corresponding protein concentration in a sample recovered from a healthy individual not at risk of developing type 1 diabetes.

In accordance with one embodiment of the present disclosure, a method of identifying a biomarker associated with early onset type 1 diabetes in humans is described. In one embodiment, the method comprises performing a first analysis of a proteome of islets from female NOD mice at three pre-diabetic time points and at a time of diabetes onset; performing a second analysis of proteome of islets from diabetic NOD mice and NOD-SCID mice that has been rendered acutely diabetic following an adoptive transfer of T cells from NOD.BDC2.5 mice; performing a third analysis of islets from NOD mice that remained diabetes-free after a time period of observations; identifying a murine biomarker based on the first, second, and third analysis; and using the murine biomarker as the biomarker associated with early onset type 1 diabetes in humans. In one embodiment, the period of observation ranges from about 46 weeks to about 48 weeks. In one embodiment, the murine biomarker is protein disulfide isomerase A1 (PDIA1) or protein 14-3-3b.

In accordance with one embodiment of the present disclosure, a method for treating subjects at risk for developing type 1 diabetes to either delay or prevent clinical insulin-dependent diabetes development is described. In one embodiment, the method comprises identifying a subject at risk of type 1 diabetes by measuring protein disulfide isomerase A1 (PDIA1) levels in a body fluid of the subject; measuring protein disulfide isomerase A1 (PDIA1) levels in a body fluid of a control; comparing the relative concentration of PDIA1 levels in the body fluid of the subject to PDIA1 levels in the body fluid of the control; and determining that the subject has type 1 diabetes when the concentration of PDIA1 levels in the blood of the subject is statistically greater than the concentration of PDIA1 levels in the control. The method further comprise administering a type 1 diabetes preventative therapeutic if the subject has type 1 diabetes.

In accordance with one embodiment of the present disclosure, a type 1 diabetes preventative therapeutic comprises restricting carbohydrate consumption by the subject, optionally with increased fluid consumption, or administration of teplizumab. In one embodiment the type 1 diabetes preventive therapeutic comprises administering a compound with immune-suppressant properties. In one embodiment the type 1 diabetes preventive therapeutic comprises administering a compound selected from the group consisting of a nonsteroidal anti-inflammatory drug, a corticosteroid, and an immune-suppressant drug. In one embodiment, the body fluid of the subject is blood or serum.

In accordance with one embodiment of the present disclosure, a method for treating a subject at risk of developing type 1 diabetes comprises measuring the concentration of multiple biomarkers that are associated with early onset type 1 diabetes. In one embodiment, in addition to measuring protein disulfide isomerase A1 (PDIA1) levels in the blood of the subject, the method further comprises measuring protein 14-3-3b levels in the body fluid of the subject; measuring protein 14-3-3b levels in the body fluid of the control; comparing the relative concentration of 14-3-3b levels in the body fluid of the subject to 14-3-3b levels in the body fluid of the control; and determining that the subject has type 1 diabetes when the concentration of PDIA1 and 14-3-3b levels in the body fluid of the subject are statistically greater than the concentration of PDIA1 levels and 14-3-3b levels in the body fluid of the control. The method comprises administering a type 1 diabetes preventative therapeutic if the subject has type 1 diabetes.

In accordance with one embodiment of the present disclosure, the method comprises identifying a subject at risk of type 1 diabetes by measuring a first biomarker level in a body fluid of the subject; measuring the first biomarker level in a body fluid of a control; comparing the relative concentration of the first biomarker in the body fluid of the subject to first biomarker level in the body fluid of the control; and determining that the subject has type 1 diabetes when the concentration of the first biomarker level in the blood of the subject is statistically greater than the concentration of the first biomarker level in the control. The method further comprises administering a type 1 diabetes preventative therapeutic if the subject has type 1 diabetes. In one embodiment, the first biomarker is associated with maintaining β cell function.

In one embodiment, the subject is a pediatric subject, and wherein the first biomarker level in the subject was measured within 48 hours of the clinical onset of type 1 diabetes. In one embodiment, the first biomarker level in the body fluid of the subject is about 10% to about 70% more than the first biomarker level in the body fluid of the control. In one embodiment, the body fluid of the subject is serum. In one embodiment, the first biomarker is PDIA1. In one embodiment, the method further comprises measuring and comparing the relative concentration of a second biomarker in the body fluid of the subject to a second biomarker level in the body fluid of the control before determining that the subject has type 1 diabetes and administering the type 1 diabetes preventative therapeutic.

In accordance with one embodiment of the present disclosure, a method for treating a subject at risk of developing a disease is described. In one embodiment, the method comprises identifying a subject at risk of the disease by measuring a biomarker level in a body fluid of the subject; measuring the biomarker level in a body fluid of a control; comparing the relative concentration of the biomarker in the body fluid of the subject to the biomarker level in the body fluid of the control; determining that the subject has the disease when the concentration of the biomarker level in the blood of the subject is statistically greater than the concentration of the biomarker level in the control; and administering a disease preventative therapeutic if the subject has the disease.

In one embodiment, the first biomarker is identified by methods comprising performing a first analysis of a proteome of tissue from female NOD mice at three pre-disease time points and at a time of disease onset; performing a second analysis of proteome of tissue from diseased NOD mice and NOD-SCID mice that has been rendered to have the disease following an adoptive transfer of T cells from NOD.BDC2.5 mice; performing a third analysis of tissue from NOD mice that remained disease-free after a time period of observations; and identifying the biomarker based on the first, second, and third analysis. In one embodiment, the disease is type 1 diabetes. In one embodiment, the biomarker is associated with maintaining β cell function.

### BRIEF DESCRIPTION OF THE DRAWINGS

FIG. 1: Schematic representation of study design and experimental workflow as described in Example 1.

FIGS. 2A-2B: Proteomic analysis of pancreatic islets in NOD and CD1 mice over time. FIG. 2A Principal component analysis (PCA) of all quantified islet proteins from NOD and CD1 mice at 10, 12, and 14 weeks of age and during diabetes onset. FIG. 2B: Heat map showing expression patterns of the top 30 differentially expressed proteins from each individual biological replicate at 10, 12, and 14 weeks of age and at diabetes onset. FIG. 2C: Unsupervised hierarchical clustering analysis of the top 30 differentially expressed proteins in NOD mice compared to age- and sex-matched CD1 mice from 10, 12, and 14 weeks of age, and during diabetes onset. (n=3-12 animals/per group).

FIGS. 3A-3B: Ingenuity pathway analysis of islet proteome. FIG. 3A: The top 10 upregulated canonical pathways changed during diabetes progression in islets collected from NOD mice compared to age-matched CD1 mice. FIG. 3B: The top 10 downregulated canonical pathways changed during diabetes progression in islets collected from NOD mice compared to age-matched CD1 mice.

FIGS. 4A-4B: Islet proteome comparison of acute and chronic models of TID. FIG. 4A: Venn diagram showing protein overlap between spontaneous and inducible models of T1D. FIG. 4B: Pathway enrichment analysis identified common pathways that are significantly activated during the pathogenesis of T1D.

FIGS. 5A-5E: Proteomics analysis of diabetes-resistant NOD mice and NOD mice that developed diabetes. FIG. 5A: Principal component analysis (PCA) of the islet proteome in NOD mice that remained diabetes free through 46-48 weeks of age (Res) and NOD mice at the time of the development of diabetes (Dev). FIG. 5B: Heat map showing top differentially expressed islet proteins in diabetes-resistant mice and mice at the time of diabetes onset. FIG. 5C: Median normalized hierarchical clustering of differentially expressed proteins. Shown are upregulated FIG. 5D and down-regulated FIG. 5E pathways (y-axis) and the corresponding number of proteins (x-axis) differentially expressed in islets from diabetes-resistant mice.

FIGS. 6A-6C: Immunofluorescence-based validation of protein targets. FIG. 6A-6C: Pancreas sections from 9, 11, and 13 weeks old NOD mice were immunostained for PDIA1, 14-3-3b, and PRDX3 (red), and co-stained with insulin (green) and glucagon (white). Scale bar=10 μm. Adjacent bar graphs show the quantitation of fluorescence intensity for each protein targets calculated using corrected total islet cell fluorescence. N=4-5 mice/age-group, *P<0.05, **P<0.001

FIGS. 7A-7D: Islet PDIA1 expression is increased in human pancreatic tissue sections from organ donors with autoantibody positivity and with TID. FIG. 7A: Human pancreatic tissue sections from non-diabetic human organ donors, organ donors who were autoantibody positive (AAb+) but without a diagnosis of T1D, and organ donors with established TID were immunostained for PDIA1 (red) and co-stained for insulin (green), glucagon (white), and DAPI (blue). Scale bar=10 mm. FIG. 7B: The bar graphs show the quantitation of fluorescence intensity for each protein target calculated using the corrected total islet cell fluorescence (n=5-7 donors/per group; 5-10 islets/donor). FIG. 7C: Western blot analysis of human islets (n=7 donors) treated with or without pro-inflammatory cytokines (IL-1β+IFNg) or high glucose (22.5 mM) for 1 hour or 24 hours, FIG. 7D: Protein expression was normalized to Revert™700 total protein staining and is presented as fold expression compared to untreated controls, all the values are presented as mean±SEM (one-way-ANOVA); *P<0.05, **P<0.001.

FIGS. 8A-8F: PDIA is increased in pre-diabetic NOD mice and in recent-onset T1D. FIG. 8A: A representative image of standard curve generated using serial dilution of recombinant PDIA1 protein showing the higher and lower sensitivity of the assay. FIG. 8B: Measurement of circulating PDIA1 in plasma samples of sex and age-matched CD1 and NOD mice with at 10 weeks. FIG. 8C: Measurement of circulating PDIA1 in plasma samples of sex and age-matched CD1 and NOD mice with at 12 weeks. FIG. 8D: Measurement of circulating PDIA1 in plasma samples of sex and age-matched CD1 and NOD mice with at 14 weeks. N=4-7 mice/group, *P<0.05, **P<0.001. FIGS. 8E-8F: Plasma concentration of PDIA1 in non-diabetic control subjects (n=10, 12+/−4.2) and patients with recent-onset TID (n=14, 11.57+/−4.05). **P=0.001, ns=not significant.

FIG. 9A-9D: Blood glucose and body weight of mice used in this study. FIG. 9A: Blood glucose levels in 10, 12, and 14 week old female NOD and CD1 mice and from NOD mice that developed diabetes and age-matched CD1 mice. FIG. 9B: Blood glucose levels in sham-operated NOD-SCID mice and NOD-SCID BDC 2.5 mice that received an adoptive transfer of T cells. FIG. 9C: Blood glucose levels in diabetes resistant female NOD mice and age-matched CD1 mice. FIG. 9D: Bar graphs showing body weight of 10, 12, and 14-week-old female NOD and CD1 mice and weight of NOD mice that developed diabetes and age-matched CD1 mice. **P<0.001; ***P<0.0005; ****P<0.0001 (t-test).

FIG. 10: Total ion current (TIC) normalization of protein intensities. FIG. 10A: Box plot of protein intensities per sample across all samples before- and after-normalization (upper and lower left panel, respectively). FIG. 10B: Density distribution plot showing the protein intensity distribution for all samples in each experimental group. This data indicates the extent of outlier samples in the proteomic data.

FIG. 11: Protein distribution across different age groups of NOD and CD1 mice. Volcano plots showing total number of identified proteins between age- and sex-matched NOD and CD1 mice at different time points.

FIG. 12A-12E: Differential protein expression patterns for all study groups. FIG. 12A: Principal Component Analysis plot with annotation. The proteomics data derived from the samples was annotated by experimental group and subject to a principal component analysis, in which the individual samples are annotated by color according to their experimental group.

FIG. 12B: Correlation matrix (Pearson r) of all the 53 proteomic samples measures from mouse islet samples. FIG. 12C: Heat map of the significant differentially expressed proteins in islet cells across all experimental comparisons, namely NOD mice relative to their age-matched CD1 controls at week 10, 12, 14, and at the onset of type 1 diabetes; NOD mice that remained diabetes-free through 46-48 weeks (res) relative to their age-matched CD1 controls; and NOD-SCID mice rendered acutely diabetic through T-cell adoptive transfer relative to their NOD-SCID controls. FIG. 12D: Heat map showing hierarchical clustering of protein peak intensities across all the study groups with protein ID's shown sequentially in columns. FIG. 12E: Heat map showing the top 75 proteins across the study groups.

FIG. 13A-13D: FIG. 13A: Upregulated Pathways in NOD mice. FIG. 13A: Pathway enrichment analysis showing significantly upregulated pathways at week 10 in NOD mice. FIG. 13B: Pathway enrichment analysis showing significantly upregulated pathways at week 12 in NOD mice. FIG. 13C: Pathway enrichment analysis showing significantly upregulated pathways at week 14 in NOD mice. FIG. 13D: Pathway enrichment analysis showing significantly upregulated pathways at the time of diabetes onset in NOD mice.

FIG. 14A-14D: Downregulated Pathways in NOD mice. FIG. 14A: Pathway enrichment analysis showing significantly downregulated pathways at week 10 in NOD mice. FIG. 14B: Pathway enrichment analysis showing significantly downregulated pathways at week 12 in NOD mice. FIG. 14C: Pathway enrichment analysis showing significantly downregulated pathways at week 14 in NOD mice. FIG. 14D: Pathway enrichment analysis showing significantly downregulated pathways at the time of diabetes onset in NOD mice.

FIG. 15A-15B: Immunofluorescence staining of ER stress markers in pancreatic tissue sections from NOD mice. FIG. 15A: Immunofluorescence staining of CHOP (red) and BIP (gray) in pancreatic tissue sections from 9, 11, and 13-week old female NOD mice (n=4-6 mice/age-group). FIG. 15B: Quantification of fluorescence intensities for BIP and CHOP. (n=4-5 mice/group (mean±SEM)); *p<0.05, **p<0.001 (one-way ANOVA).

### DETAILED DESCRIPTION

In describing and claiming the methods, the following terminology will be used in accordance with the definitions set forth below.

The term “about” as used herein means greater or lesser than the value or range of values stated by 10 percent, but is not intended to designate any value or range of values to only this broader definition. Each value or range of values preceded by the term “about” is also intended to encompass the embodiment of the stated absolute value or range of values.

As used herein, the terms “effective amount” or “therapeutically effective amount” of a compound refers to a nontoxic but sufficient amount of the compound to provide the desired effect. The amount that is “effective” will vary from subject to subject, depending on the age and general condition of the individual, mode of administration, and the like. Thus, it is not always possible to specify an exact “effective amount.” However, an appropriate “effective” amount in any individual case may be determined by one of ordinary skill in the art using routine experimentation.

As used herein, the term “subject” means an animal including but not limited to humans, domesticated animals including horses, dogs, cats, cattle, and the like, rodents, reptiles, and amphibians.

As used herein, the term “patient” without further designation is intended to encompass any warm blooded vertebrate domesticated animal (including for example, but not limited to livestock, horses, cats, dogs, and other pets) and humans receiving a therapeutic treatment whether or not under the supervision of a physician.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

As used herein, the term “treating” includes alleviation of the symptoms associated with a specific disorder or condition and/or preventing or eliminating said symptoms.

As used herein, the term “biomarker,” “marker,” or “molecular marker,” is a biological molecule found in blood, urine, other body fluids such as lymph fluid or breast milk, or tissues that is a sign of a normal or abnormal process, or of a condition or disease. A biomarker may be a protein, a peptide, a gene, a cytokine, a metabolite, a cell, or any other biologically relevant material. A biomarker may be used to see how well the body responds to a treatment for a disease or condition. A biomarkers may be used to predict a disease, predict an early onset of disease, or to predict relevant clinical outcomes across a variety of treatments and populations. These substances can be found in the blood, urine, stool, tumor tissue, serum, or other tissues or bodily fluids of patients. In particular here, the biomarkers are found in blood or serum.

In one embodiment, the biomarker may indicate a disease state in the patient. In one embodiment, the disease is an autoimmune disease. In one embodiment, the autoimmune disease is diabetes. In one embodiment, the disease in type-1 diabetes.

In one embodiment, a method can be employed to identify a biomarker indicating the type 1 diabetes. In one embodiment, the method further comprises monitoring the patient for type 1 diabetes. In one embodiment, the method further comprises determining if the patient is eligible for a preventative therapeutic for type 1 diabetes.

In one embodiment, administering the preventative therapeutic comprises administering the patient with a drug. In one embodiment, administering the preventative therapeutic comprises administering the patient with a therapeutic regimen that affects patient behavior including but not limited to altering diet or fluid intake.

As disclosed herein, temporal changes in islet cell protein expression during the evolution of T1D have been investigated using three mouse models of T1D and high-throughput, discovery-scale SWATH-MS proteomics.

In accordance with one embodiment of the present disclosure, longitudinal analysis of pancreatic islets of female NOD mice during the progression to TID enabled identification of stress pathways that are activated prior to β cell destruction. The identification of such stress pathways can be utilized to identify clinical biomarkers and develop potential therapeutics.

In accordance with one embodiment of the present disclosure, a core set of pathways that are essential for beta cell health and function have been identified that are modulated in a temporal fashion during the development of T1D. Key findings were validated using immunofluorescence in tissue sections from NOD mice.

In accordance with one embodiment, the present disclosure is directed to a study aimed at identifying temporal changes in islet β cell protein expression during the evolution of TID using three distinct mouse models of TID and high-throughput, discovery-scale SWATH-MS proteomics. The proteome of islets collected from female NOD mice at three pre-diabetic time points and at the time of diabetes onset was analyzed and protein abundance was compared with sex- and age-matched non-diabetic CD1 mice. To compare chronic and acute models of TID, the proteome of islets from diabetic NOD mice and NOD-SCID mice that has been rendered acutely diabetic following the adoptive transfer of T cells from NOD.BDC2.5 mice was analyzed. Finally, to gain insight into potential mechanisms that contribute to β cell resiliency in the face of immune activation, the islet proteomes of NOD mice at diabetes onset was compared to NOD mice that remained diabetes-free after about 46 to about 48 weeks of observation.

In accordance with one embodiment, the proteome of islets collected from female NOD mice at three pre-diabetic time points and at the time of diabetes onset were analyzed. The protein abundance with sex- and age-matched non-diabetic CD1 mice were compared to each other. To compare chronic and acute models of TID, the proteome of islets from diabetic NOD mice and NOD-SCID mice that has been rendered acutely diabetic following the adoptive transfer of T cells from NOD.BDC2.5 mice were analyzed. Finally, to gain insight into potential mechanisms that contribute to β cell resiliency in the face of immune activation, the islet proteomes of NOD mice at diabetes onset were compared to NOD mice that remained diabetes-free after 46-48 weeks of observation.

In accordance with one embodiment, analysis of these three models revealed several notable themes. In the dataset obtained from the longitudinal NOD cohort, an early but time-restricted increase in the expression of several proteins previously linked with secretory function, proinsulin folding, and stress mitigation, including proteins known to be involved in the mitigation of endoplasmic reticulum and oxidative stress was observed. Interestingly, week 14 was observed as a potential inflection point, where the loss of expression of these protective proteins heralded TID onset. Consistent with this, pathways reflecting mitochondrial dysfunction, UPR activation ER stress, mTOR signaling, and cell: cell communication were upregulated, whereas several metabolic pathways, including oxidative phosphorylation, fatty acid oxidation, and glutathione metabolism, and phagosome maturation and ubiquitin signaling, were down-regulated during T1D progression.

In accordance with one embodiment, this pattern is reminiscent of metabolic data from natural history cohorts of autoantibody positive individuals who progress to TID, where there are compensatory changes in the architecture of insulin secretion that largely maintain glycemia until ˜12 months prior to disease onset, followed by marked loss of insulin secretion and rapidly worsening glycemic control until diabetes diagnosis. The findings are consistent with cross-sectional studies that have analyzed gene and protein expression patterns in pancreatic sections from human donors with diabetes and in previous studies in mouse models of diabetes, where a prominent role for ER and mitochondrial dysfunction has been identified during disease progression. Notably, these pathways were found to be activated early in the disease process and it was observed that there was overlap between several of these key activated stress pathways in the NOD mouse model and in the acute, inducible model of T1D. While the former is a spontaneous and chronic model and the latter is an acute model of islet destruction, similarities between the proteomic analyses of these two models highlight the importance of this core set of pathways during the development of T1D.

In accordance with one embodiment, to validate selected findings from the proteomics analysis, immunofluorescence analysis of three targets identified in the longitudinal NOD cohort was performed: 14-3-3B, PRDX3, and PDIA1. Members of the 14-3-3 protein family have been implicated in various metabolic signaling pathways and have been linked with protection against apoptosis in pancreatic β cells. PRDX3 prevents mitochondrial dysfunction, and its overexpression is protective against oxidative stress induced by insulin resistance and hyperglycemia. PDIA1 is a highly abundant ER-localized thiol oxidoreductase that has been implicated in glucose-stimulated insulin secretion, proinsulin processing, and protection against ER stress.

PDIA1 has been described as a secreted protein, and in other cell types, PDIA1 release is increased in the setting of injury and stress. Extracellular PDIA1 has been linked with the regulation of thrombus formation during vascular inflammation, but a complete understanding of the extracellular role of this protein is lacking. Interestingly, anti-PDIA1 antibodies have been identified in patients with recent-onset TID, suggesting that β-cell derived PDIA1 serves as a TID autoantigen. Against this background, in accordance with one embodiment, it was hypothesized that increased β cell expression of PDIA1 may be reflected in the circulation and that measurement of PDIA1 may have utility as a TID biomarker. To test this possibility, a high-sensitivity electrochemiluminescence assay was developed to measure serum and plasma PDIA1. Using this assay, an increase in plasma PDIA1 in pre-diabetic NOD mice and in the serum of children with recent-onset T1D was documented. This disclosure represents the first assessment of circulating PDIA1 in individuals with diabetes.

In accordance with one embodiment of the present disclosure, the utility of the approach of prioritizing TID biomarkers has been validated using PDIA1. PDIA1 is one protein that was differentially expressed in islets from NOD mice and was predicted to be secreted as a target for the development of a high sensitivity electrochemiluminescence assay using Meso Scale Discovery technology. PDIA1 is a protein known to play an essential role in proinsulin processing and folding, and insulin secretion. Using this approach, increased β-cell expression and serum levels of PDIA1 was validated in NOD mice during the evolution of T1D and in the serum of children with recent onset TID. In accordance with one embodiment of the present disclosure, PD1A1 may serve as a biomarker of type 1 diabetes.

### Example 1: Animals and Experimental Procedures

Female NOD/ShiLTJ (NOD), a model of spontaneous T1D development, were purchased from the Jackson Laboratory. NOD-BDC2.5 and NOD-SCID mice were used in the adoptive transfer experiments and were also purchased from the Jackson Laboratory. Female outbred CD1 mice were purchased from Charles River Laboratories. Mice were maintained at the Indiana University School of Medicine Laboratory Animal Resource Center under pathogen-free conditions and protocols approved by the Indiana University Institutional Animal Care and Use Committee.

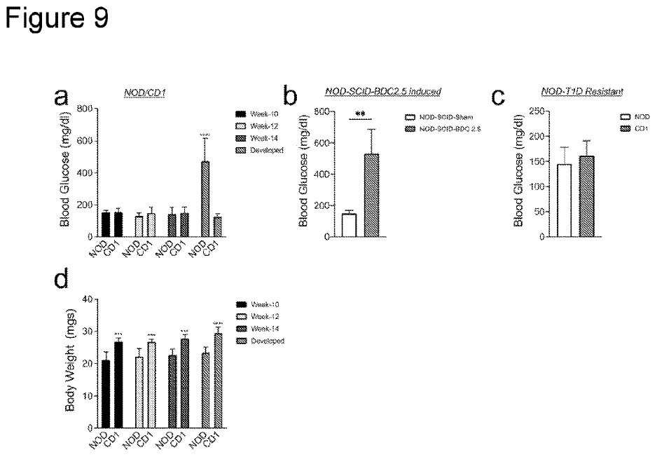

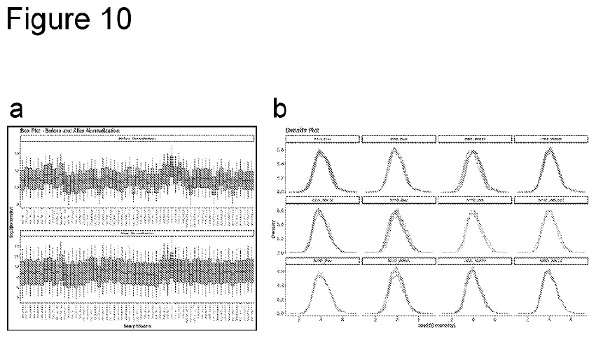

Mice were allowed to acclimate for at least two weeks upon arrival and before the initiation of experiments. Blood glucose was monitored weekly in all the mouse models and diabetes was defined as a blood glucose >250 mg/dL for two consecutive measurements. Blood glucose and body weight were recorded on the day of islet isolation for each age group of mice used for downstream analysis (FIG. 9A-D). At the indicated time points, pancreatic islets were isolated, or the pancreas was harvested. Islets were hand-picked, washed twice with PBS, and stored in pellets at −80° C. until use. For plasma collection, blood was obtained at the time of euthanasia via cardiac puncture, transferred to a Becton Dickinson Vacutainer K2EDTA tube (Cat #365974), and centrifuged at 5,000 rpm for 10 minutes at 4° C. The separated plasma samples were aliquoted into 1.5 ml cryotubes and stored at −80° C. until use.

For adoptive transfer experiments, single-cell splenocyte suspensions were prepared from 12-week-old male NOD-BDC2.5 mice. CD4+ T cells were purified by negative selection (Cat #558131, BD Biosciences), activated in 6-well plates (5×10⁶ cells/well) coated with anti-CD3 and anti-CD28 (1 mg/mL each), and expanded for 72 h in T-75 flasks (5×10⁶ cells/flask) in complete RPMI 1640 medium (1% penicillin/streptomycin and 10% FBS) containing 100 U/mL IL-2. Cells were then collected, washed twice with Hanks' balanced salt solution (HBSS), and diluted to 5×10⁶ cells/mL in HBSS. Recipient 8-week-old immunodeficient male NOD-SCID mice received 1×10⁶ T cells via intraperitoneal injection, and blood glucose was measured daily for 21 days. Age-matched NOD-SCID mice that received HBSS alone were used as controls. The onset of diabetes was defined as two consecutive blood glucose readings of ≥275 mg/dL.

### Example 2: Mass Spectrometry Sample Processing

Islet pellets were lysed by adding 48 mg of urea to ˜100 μL of pelleted cells. Lysates were ultrasonicated by 5 successive 10 s pulses to ensure complete lysis and to shear DNA. After determining protein content using a BCA assay, 50 μg of protein was transferred to a 1.5-ml tube, and the volume was adjusted to 250 μL using 50 mM ammonium bicarbonate (pH 8.0). The sample was then reduced (fresh tris(2-carboxyethyl) phosphine, 25 mM at 37° C. for 40 min), alkylated (fresh iodoacetamide, 10 mM for 40 min at room temperature (RT) in the dark), and diluted to 800 μL with 50 mM ammonium bicarbonate. The pH of the sample was adjusted to 8.0, and tryptic digestion was performed at 37° C. overnight in the presence of 10% acetonitrile with constant agitation, using trypsin at a 50:1 ratio. The digest was then acidified with 10% FA (pH 2-3), desalted on a 96-well HLB microelution plate, and dried before mass spectrometry (MS) analysis.

### Example 3: Data-Independent Acquisition LC-MS/MS Analysis

Two μg of peptides were injected onto a liquid chromatography (LC) column and analyzed by LC-MS/MS on a SCIEX 6600 TripleTOF mass spectrometer (SCIEX, Framingham, MA) operated in data-independent acquisition (DIA) mode. Peptides were loaded onto an Eksigent 415 HPLC system equipped with an Ekspert nanoLC 400 autosamplers. Peptides were separated using a ChipLC trap-elute system equipped with a 15-cm, 75-μm inner-diameter C18 column (300 Å diameter) at a flow rate of 500 nL/min using a linear AB gradient of 3-35% solvent B (0.1% FA in acetonitrile) for 60 min, 35-85% B for 2 min, hold at 85% B for 5 min, and re-equilibration at 3% B for 7 min. Mass spectra were obtained with 64 variable-width precursor isolation windows. Dwell-times in MS1 and MS2 were 250 and 45 ms, respectively, for a total cycle time of 3.2 s. The collision energy was optimized for an ion m/z centered on the isolation window, with the collision energy spread ranging from 5-15. Source gas 1 was set to 3, gas 2 was set to 0, curtain gas was set to 25, source temperature was set to 100° C., and source voltage was set to 2400 V.

### Example 4: Post-Acquisition Analysis

Peptide library generation. Individually acquired DIA files were processed using the Signal Extraction module of the DIA-Umpire software tool (DIAu-SE). Pseudospectra generated in the DIAu-SE step was then processed for library generation. Mouse protein sequences were defined in a FASTA database of the Swiss-Prot-reviewed canonical mouse genome appended with Biognosys iRT peptides for retention time alignment (Biognosys, Schlieren, Switzerland) and randomized decoy sequences.

### Example 5: Quantitation of Individual Specimen DIA-MS Files

Raw intensity data for peptide fragments were extracted from DIA files using the open-source openSWATH workflow against the sample-specific peptide assay library described above. Briefly, peptide assay peak groups were extracted from raw DIA files and scored against an equal number of decoy peak groups based on a composite of 11 data-quality subscores. Target peptides with a false-discovery rate of identification <1% were included for downstream analyses.

### Example 6: Curating Files for Quality

All files were individually curated prior to protein-level roll-up and subsequent quantitation. The following parameters were considered: total ion chromatogram profile and intensity, file quality within the library build (Q1, Q2, Q3 data distribution from DIAumpire), and raw distribution of proteins compared to decoys derived in openSWATH. Files exhibiting aberrant or low-quality results for any of these parameters were excluded from subsequent analysis steps. All steps were performed while blinded to filenames or experimental group.

### Example 7: Data Normalization, Protein-Level Roll-Up, and Statistical Analyses

The total ion current associated with the MS2 signal across the chromatogram was calculated for normalization, excluding the last 15 min to avoid including the signal from contaminants/noise. This ‘MS2 signal’ of each file, akin to a total protein load stain on a Western blot gel, was used to adjust each transition intensity of each peptide in the corresponding file. Normalized transition-level data were then processed using mapDIA software to remove noisy/interference transitions from the peptide peak groups, calculate peptide and protein level intensities, and perform pairwise comparisons between groups.

The following pairwise comparisons were made: NOD vs. CD1 for each time point (weeks 10, 12, and 14); NOD-BDC2.5 vs. NOD-SCID ctrl; NOD resistant vs. NOD mice with diabetes. The mapDIA tool generates a q-value to indicate a false-discovery rate rather than a simple p-value. It was assumed that protein expression differs significantly between two groups when the log 2 (fold-change) was >0.6 (i.e., ˜1.5 fold-change) and the q-value/false-discovery rate was <0.01.

### Example 8: Immunofluorescence Staining and Quantification

Immunofluorescence (IF) was performed to investigate key findings from the MS analysis. Briefly, formalin-fixed paraffin-embedded (FFPE) pancreatic tissues from an independent cohort of pre-diabetic age-matched NOD mice, obtained at 7, 9, 11, 13 weeks of age and mice that developed diabetes, were sectioned at a thickness of 5 mm and deparaffinized. The sections were hydrated twice with fresh Xylene for 5 minutes and a series of decreasing ethanol concentrations (100 to 70%). Antigen retrieval was performed using citrate buffer and stained using antibodies against PDIA1 (Cell Signaling, Cat #3501S, RRID: AB_2156433), PRDX3 (Abcam, Cat #ab73349, RRID: AB_1860862), 14-3-3 B/YWHAB (Sigma, Cat #HPA011212, RRID: AB_1844334), insulin (Dako, Cat #IR002, RRID: AB_2800361), glucagon (Abcam, Cat #ab10988, RRID: AB_297642), CHOP (ThermoFisher Scientific, Cat #MA1-250, RRID: AB_2292611) and BIP (Cell Signaling Technology, Cat #3177S, RRID: AB_2119845). Similarly, human pancreatic tissue sections from non-diabetic cadaveric organ donors, organ donors with autoantibody positivity but no diabetes, and organ donors with TID were received from the Network of Pancreatic Organ Donors (nPOD) Biorepository and stained for PDIA1, insulin, and glucagon using the above-mentioned primary antibodies. Secondary antibodies included Alexa 488-labeled goat anti-guinea pig, Alexa 568-labeled donkey anti-rabbit, and Alexa 647-labeled donkey anti-mouse antibodies. Nuclei were stained with DAPI. Images were acquired using an LSM800 confocal microscope (Zeiss, Germany) and quantified using Image-J software (NIH) as described previously (31). From each mouse (4-7 mice/group), 3-7 islets were randomly selected for imaging, and for human pancreatic sections, 5-10 islets from every donor were randomly selected for imaging. Normalized total islet cell fluorescence intensity was calculated independently by two individuals working in a blinded fashion.

### Example 9: Human Pancreatic Islet Culture and Treatment

Human pancreatic islets were received from the Integrated Islet Distribution Program (IIDP) and recovered overnight in a complete Prodo culture medium (Prodo, Cat #PIM S001GMP). The next day, islets were replenished with fresh medium with or without 1000 U/mL of IFNg and 50 U/mL of IL-1ß (R&D Systems, Cat #285-IF-100; Cat #201-LB-005) or with 22.5 mM of glucose for 1 hour or 24 hours. After the indicated treatment, human islets were handpicked, washed with 1×PBS, and used for downstream applications.

### Example 10: Western Blot Analysis

Human islets were lysed with lysis buffer and protein concentrations were measured by the Lowry method as described previously (2); 20 mg of protein was electrophoresed using 4-12% Bis-Tris Plus gel (Invitrogen, Cat #NW04122BOX) as per the manufacturer instructions. The proteins were transferred onto a PVDF membrane using 1×NuPAGE transfer buffer at 15 V for 1 hr. The membranes were rinsed with ddH2O for 30 seconds and stained with Revert™ 700 Total Protein stain (LI-COR, Cat #D20203-01). The membranes were washed with Revert™ 700 wash solution and imaged using ODYSSEY CLx (LI-COR) system. Then, the membranes were rinsed with ddH2O 5× times, blocked with Intercept Blocking Buffer (LI-COR, Cat #927-70001) for 45 minutes, and incubated with primary antibodies for IRE1α (Cell Signaling, Cat #3294S, RRID: AB_823545), BIP (Cell Signaling, Cat #3177S, RRID: AB_2119845), and PDIA1 (Thermo Fisher, Cat #MA3-019, RRID: AB_2163120) overnight at 4° C. The membranes were washed with 0.05% PBS-Tween and blocked with blocking buffer for 45 minutes. Then the blots were incubated with Goat anti-rabbit or Goat anti-mouse secondary antibodies (LI-COR, Cat #926-68071 RRID: AB_10956166); Cat #926-68070, (RRID: AB_10956588) for 2 hour at RT and imaged using the ODYSSEY CLx (LI-COR) imaging systems. Protein expression was quantified using Image Studio (LI-COR) and analyzed using GraphPad Prism 9.

### Example 11: Collection of Human Serum Samples

Fasting serum was obtained from children with recent-onset TID and age and sex-matched non-diabetic healthy controls under protocols approved by the Indiana University Institutional Review Board. Written informed consent or parental consent and child assent were obtained from all participants. Children with TID had been newly diagnosed within 48 hours of blood collection and were hospitalized at the Riley Hospital for Children. Non-diabetic pediatric control subjects were ambulatory, did not take any chronic prescription medications, and were free of any chronic or acute illness within two weeks preceding sampling.

### Example 12: Assay Development for Measurement of Serum PDIA1

To measure circulating levels of PDIA1, a high-sensitivity electrochemiluminescence assay was developed using the Meso Scale Discovery (MSD) ELISA conversion kit (Cat #K15A01-1), according to the manufacturer's instructions. Briefly, five anti-P4HB/PDIA1 antibodies were purchased from multiple vendors and screened for their ability to bind human recombinant PDIA1 protein (rPDIA1). The day before the experiment, single spot standard plates of the conversion kit were washed three times with 150 μL of PBS and incubated overnight with 30 μL of each antibody in PBS at 4° C. (27,28). The following day, the antibodies were washed with 0.05% PBS-Tween 20 (PBS-T) and blocked with 1% of blocking buffer A (cat #R93BA-1) for 1 hour in an orbital shaker at 700 rpm. A 4-fold serial dilution of rPDIA1 was prepared with a starting concentration of 2500 ng/mL, which was added to the plates and incubated in an orbital shaker for 1 hour at RT. Then, the plates were washed three times with 0.05% PBS-T and incubated with a PDIA1 detection antibody generated from different species (for example, mouse capture antibody was used with rabbit detection antibody) to prevent cross-reactivity and in an orbital shaker for 1 hr. Subsequently, the plates were washed three times with 0.05% PBS-T and incubated with species specific Sulfo-Tag for 1 hour in an orbital shaker. Next, the plates were washed three times with 0.05% PBS-T, 150 μL of 1× read-buffer (Cat #R92TC-2) was added to each well, and the signal was detected immediately using a MESO QuickPlex SQ 120 plate reader (MSD). Data were analyzed using Discovery Workbench software version 4.0.

### Example 13: Validation of PDIA1 Measurement in Serum and Plasma Samples

To quantitate circulating levels of PDIA1 in human serum and mouse plasma samples, standard one spot MSD plates were incubated with 5 μg/mL of capture antibody (Cat #HPA018884; Sigma) overnight at 4° C., and the same procedures described above under “Assay development” were followed. Thirty microliters of 2-fold diluted mouse plasma samples or thirty microliters of 4-fold diluted human serum samples were assayed. At the same time, 4-fold serially diluted rPDIA1 protein with a starting concentration of 2500 ng/mL was used to generate a standard curve. Following sample incubation, plates were washed as described above and incubated with mouse PDIA1 detection antibody (Cat #MA3-019; Thermo Fisher Scientific) for 1 hour in an orbital shaker at (RT). The plates were then washed and incubated with an MSD mouse Sulfo-Tag for 1 hour at RT in a shaker. Finally, the plates were read using 150 μL of read-buffer in a Quick Plex SQ 120 plate reader (MSD), and the data were analyzed as described above.

### Example 14: Statistical Analysis

The statistical analysis of the proteomics data as detailed above. Other experimental data were analyzed using GraphPad Prism version 9. Statistical significance of the difference between two groups was determined using the Student's t-test and between more than two groups by one-way ANOVA; p≤0.05 was considered significant. Data are presented as mean±S.E.M or mean±S.D.

### Example 15: Analysis of Temporal Changes in the NOD Proteome During Disease Progression

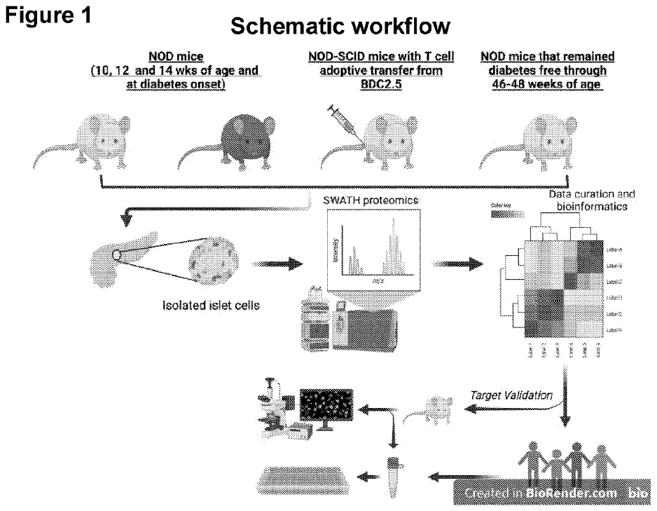

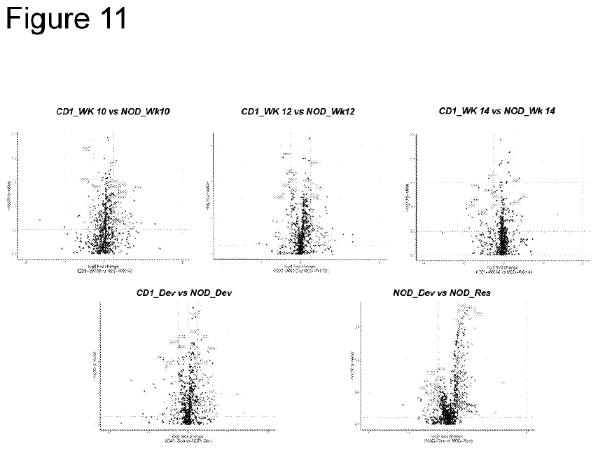

To characterize temporal changes in islet protein expression during diabetes progression, pancreatic islets were isolated from age-matched CD1 and NOD mice at 10, 12, and 14 weeks of age and at the time of diabetes onset (mean age of diabetes development 17±3.3 weeks; mean±S.D.) and analyzed using LC MS/MS (FIG. 1). FIG. 9A-D show the blood glucose and body weight data for mice on the day of islet isolation. An average of 1160 proteins and 897 overlapping proteins were quantified in NOD and CD1 mouse islets (FIG. 11). Since CD1 mice are not diabetes-prone and exhibit tightly regulated blood glucose levels, sex- and age-matched CD1 mice were used to normalize protein abundance in NOD islets. To identify differentially expressed proteins at each time point, results were analyzed using median normalization, a filtering criterion of a 1.5-fold change in protein abundance, and p≤0.05.

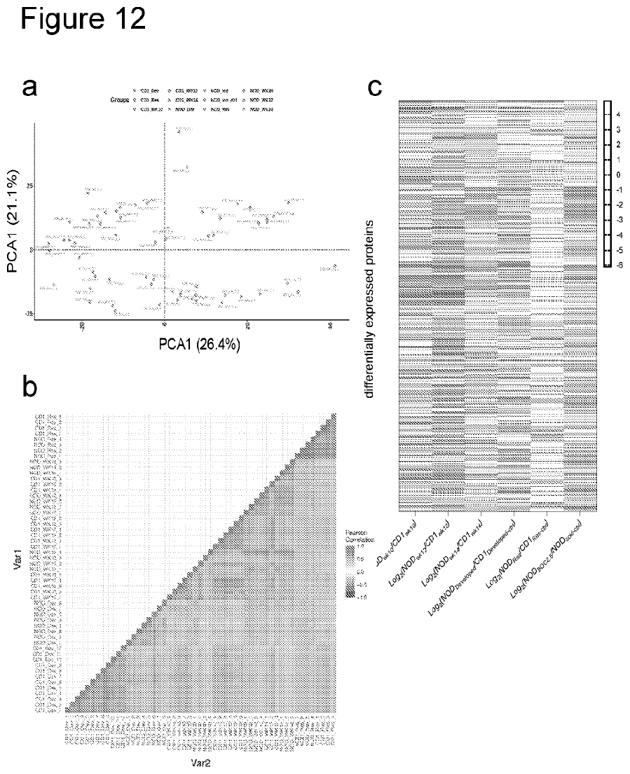

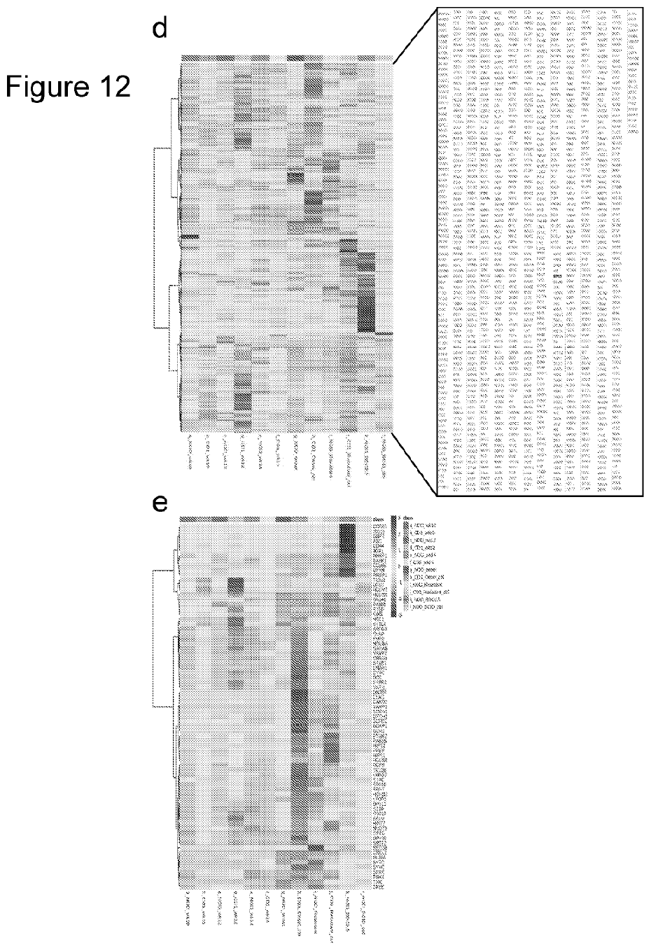

As shown in FIG. 12A-B, biological replicates across each experimental group clustered together, indicating the reproducibility of the proteomics approach. FIG. 12 C-E illustrate patterns of differentially expressed proteins and hierarchical clustering between the experimental groups. 411 up-regulated and 502 down-regulated proteins were identified in islets from 10 week old NOD mice; 364/524 at week 12 and 530/220 at week 14. A total of 344 up-regulated and 584 down-regulated proteins were identified in islets from NOD mice at the time of diabetes development. Similar results (434/275) were observed in islets from NOD-SCID-BDC2.5 mice rendered acutely diabetic. Finally, a total of 428 up- and 474 down-regulated proteins were identified in islets from the diabetes-resistant NOD mice. An average of 1160 proteins was quantified in NOD and CD1 mouse islets (FIG. 9A-D).

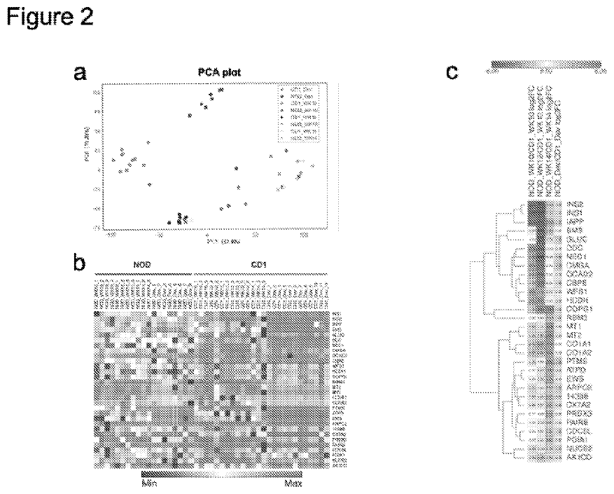

In principal component analysis (PCA), proteins from NOD mice at different pre-diabetic ages (10, 12, and 14 weeks) clustered primarily as one group, whereas proteins from diabetic mice were distinctly clustered (FIG. 2A). The top 30 differentially expressed (DE) proteins from individual mice are shown in FIG. 2B. Unsupervised hierarchical clustering analysis of these DE proteins revealed a time-dependent loss of insulin and islet amyloid polypeptide (IAPP) expression (FIG. 2C). As expected, there was a time-dependent loss of insulin and islet amyloid polypeptide (IAPP) expression. Interestingly, a time-restricted upregulation of several proteins implicated in stress mediation and normal β cell function maintenance was observed. Proteins exhibiting this pattern of expression included actin-related protein 2/3 complex 2 (ARPC2), which regulates actin cytoskeleton-mediated transport of secretory vesicles (29-31), collagen 1A1 (COL1A1), and collagen 1A2 (COL1A2), which are extracellular matrix proteins, and the metallothioneins MT1 and MT2, which have been linked with suppression of immune responses. A similar pattern was observed for peroxiredoxin 3 (PRDX3), a protein that has been linked with the regulation of mitochondrial function, and 14-3-3b, which plays a role in a number of metabolic processes, including mTOR signaling, amino acid metabolism, and mitochondrial function. Protein disulfide isomerase A1 (PDIA1) was upregulated similarly during weeks 12 to 14. Notably, PDIA1 is a thiol reductase that plays a critical role in proinsulin folding and regulation of ER function. Overall, this pattern of upregulation was observed through the 14-week time point, followed by a declining pattern expression of several of these potentially protective proteins by the time of diabetes development (FIG. 2C).

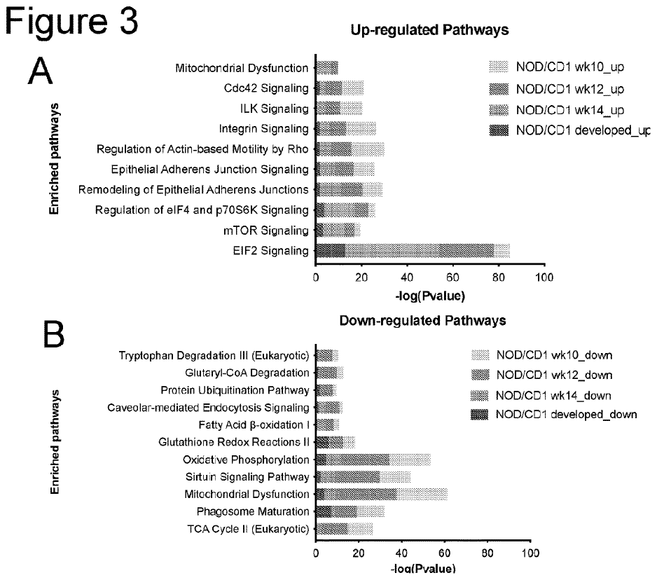

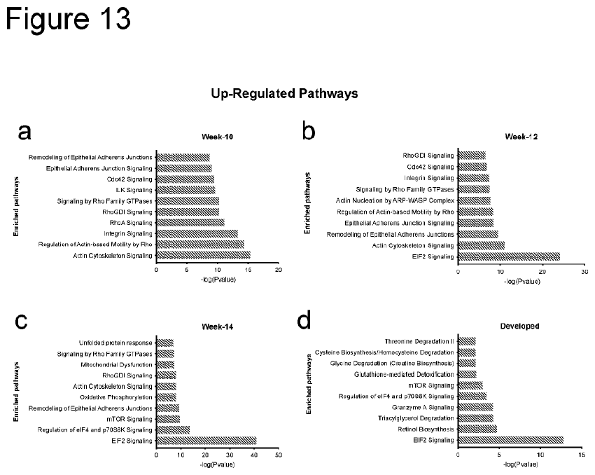

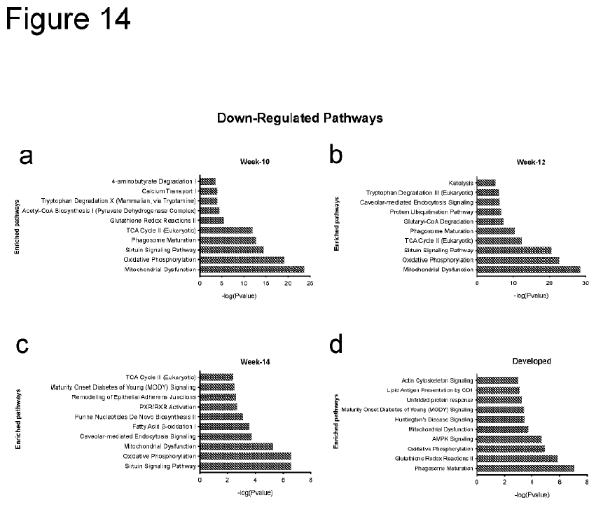

FIG. 3A shows the top 10 upregulated pathways, while FIG. 3B shows the top 10 downregulated pathways from the longitudinal analysis of NOD mouse islets. During diabetes progression, pathways related to Cdc42, integrin signaling, actin, epithelial adherens, and mTOR signaling were all upregulated (FIG. 3A). EIF2 signaling, which is associated with the unfolded protein response and ER stress, was markedly upregulated at weeks 12 and 14 and in diabetic mice (FIG. 3A). Changes in mitochondrial function were represented in both up and down down-regulated pathways. Among the significantly downregulated pathways were several metabolic pathways, including the TCA cycle, oxidative phosphorylation, fatty acid oxidation, and glutathione redox reactions. Sirtuin signaling and phagosome maturation were also downregulated (FIG. 3B and FIGS. 13-14).

### Example 16: Comparison of Acute and Chronic Models of Immune Activation

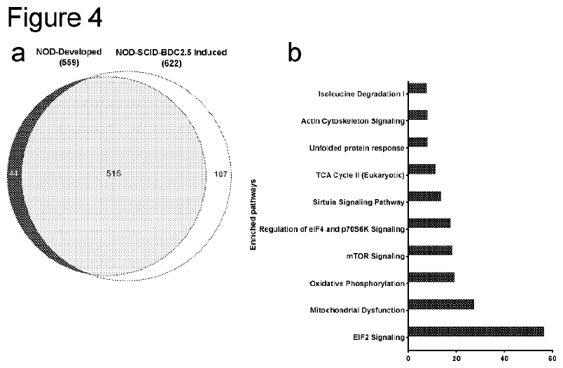

To identify commonalities and differences in the islet proteome between an aggressive, acute model of immune-mediated β cell destruction and the chronic progressive NOD model, proteomics results from islets isolated from diabetic NOD mice and islets isolated at the time of diabetes development from NOD-SCID mice that had undergone adoptive transfer of CD4+ T-cells from NOD.BDC2.5 mice was compared. Mice in the latter group develop significant hyperglycemia around 7 days following adoptive transfer. Despite this difference in the time-course of diabetes development compared to the chronic and slowly progressive NOD model, approximately ˜65% of identified proteins were common to both models (FIG. 4A). In addition, a comparison of functional canonical pathways suggested that similar pathways were activated in both models. Key overlapping pathways included modulation of EIF2 signaling and the unfolded protein response, mitochondrial dysfunction, oxidative phosphorylation, and mTOR signaling (FIG. 4B). These results suggest that irrespective of the type of inflammation (acute or chronic), similar patterns of β cell stress are activated, underscoring the importance of this core set of pathways in TID pathogenesis.

### Example 17: Proteome Comparison of NOD Mice that Developed Diabetes and Those Remaining Diabetes-Free

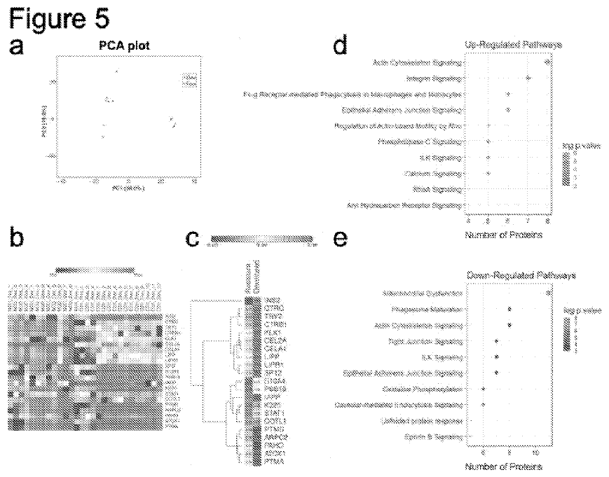

It was reasoned that comparing diabetic NOD mice and NOD mice that remained diabetes-free through extended follow-up might identify protective pathways within the β cell during immune activation. Therefore, proteomic analysis was performed on islets from 46 weeks to 48 weeks old NOD mice who remained diabetes free. Results were compared to islets collected from NOD mice at the time of diabetes development. To account for differences that may be driven by age, data from each NOD group was normalized to their respective CD1 age-matched controls. Compared to NOD mice that developed diabetes, NOD mice that remained diabetes free had markedly fewer proteins that were differently expressed relative to their age-matched CD1 controls (FIG. 12A). Principal component analysis indicated a clear separation between the diabetes-resistant group and diabetic NOD mice (FIG. 5A). Data from individual biological replicates is shown in FIG. 5B. Next, unsupervised hierarchical clustering analysis was performed using the Euclidian distance and average linkage method (FIG. 5C). Data from this analysis revealed upregulation of several unique proteins previously linked with the mitigation of β cell stress and maintenance of normal β cell function in the diabetes resistant NOD mice.

Among the top proteins that were upregulated in diabetes resistant mice and down-regulated in diabetic mice were IAPP and antioxidant-1 (ATOX1), a copper chaperone shown to be protective against hydrogen peroxide and superoxide mediated-oxidative stress. Other key proteins showing this pattern of expression were proteasome subunit beta 10 (PSB10), which is involved in the maintenance of protein homeostasis, coactosin like protein (COTL1), an F-actin-binding protein that plays a role in cellular growth; and S100A4, which functions as an intracellular cytosolic calcium sensor.

Pathway enrichment analysis was performed to uncover the signaling pathways that were differentially regulated between these two groups. FIG. 5D shows the top 10 significantly upregulated pathways in diabetes-resistant NOD mice compared to diabetic NOD mice. The size of each circle indicates the number of proteins enriched in each pathway, and the density of each circle represents their p-values. In diabetes resistant NOD mice, there was significant upregulation of pathways involved in maintaining cellular homeostasis, tissue repair, tissue clearance (i.e., phagocytosis in macrophages and monocytes), and aryl hydrocarbon receptor signaling, which has been linked with the mitigation of insulitis in NOD mice. In line with this, downregulation of pathways related to mitochondrial dysfunction, phagosome maturation, oxidative phosphorylation, and the unfolded protein response was observed (FIG. 5E). Interestingly, actin cytoskeleton signaling and epithelial adherens junction signaling were identified as being among both the up- and downregulated pathways, with distinct proteins implicated within the up and down regulated categories.

### Example 18: Analysis of Protein Targets by Immunofluorescence and Immunoblot

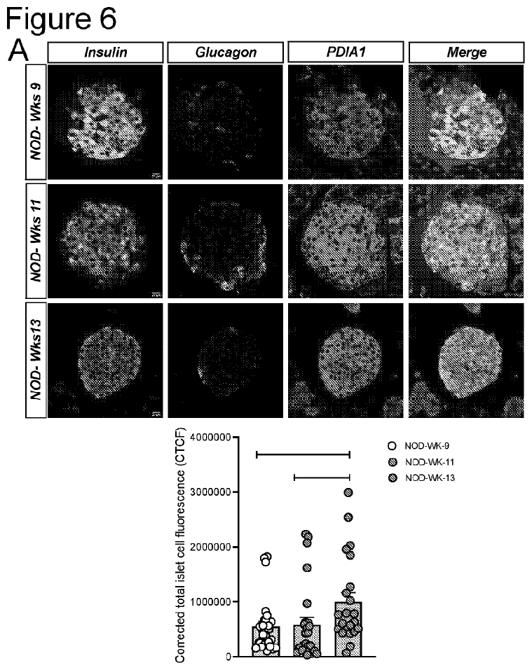

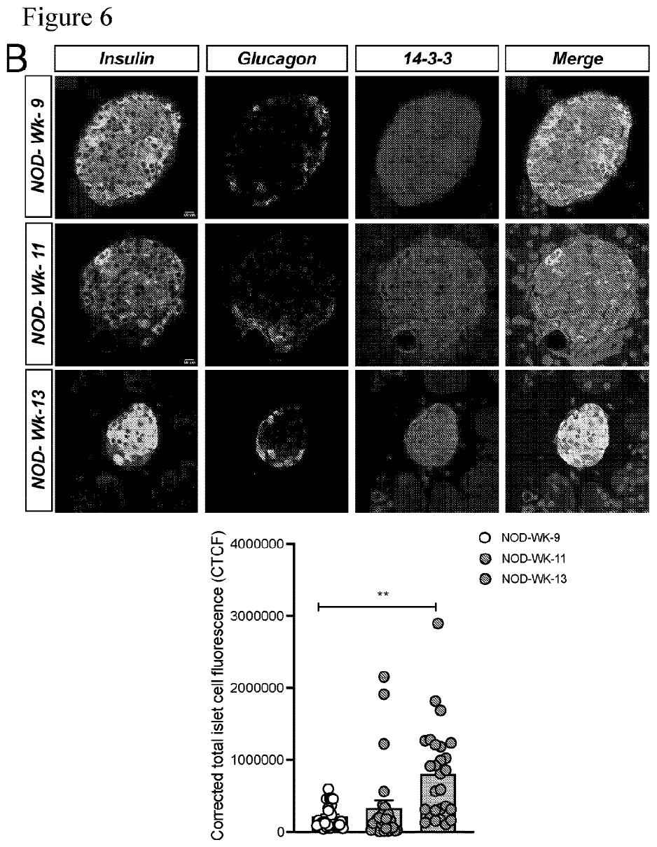

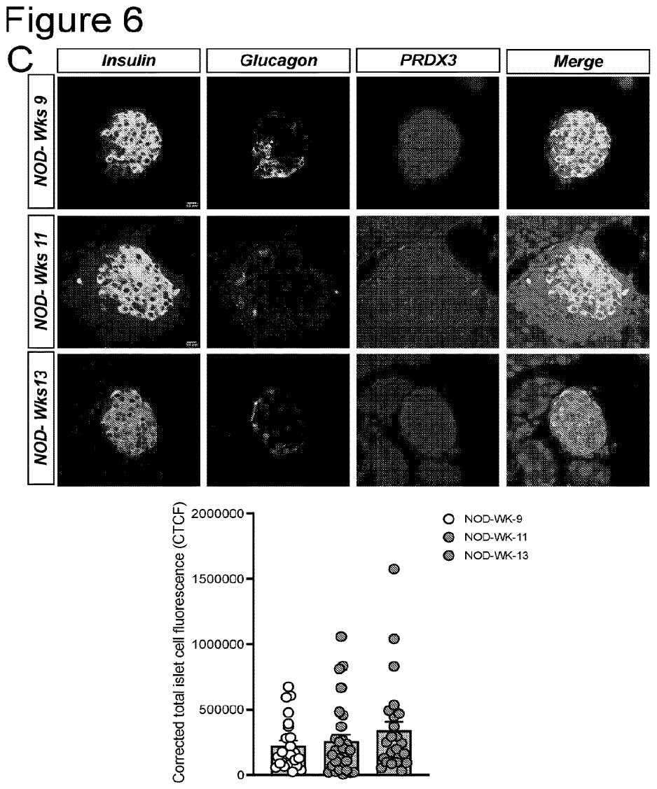

To validate key findings from the proteomic analysis, immunofluorescence was performed in pancreatic sections collected from a separate cohort of NOD mice aged 9 to 13 weeks. Three protein targets, PDIA1, 14-3-3b, and PRDX3, were selected for validation experiments based on top hits from the analysis shown in FIG. 2B and their known roles in maintaining β cell function. Consistent with proteomics data, the staining intensity of PDIA1 (FIG. 6A) and 14-3-3b in β cells significantly increased from 9 weeks to 13 weeks of age in NOD mice (FIG. 6B), while changes in the staining intensity of PRDX3 did not reach statistical significance (FIG. 6C).

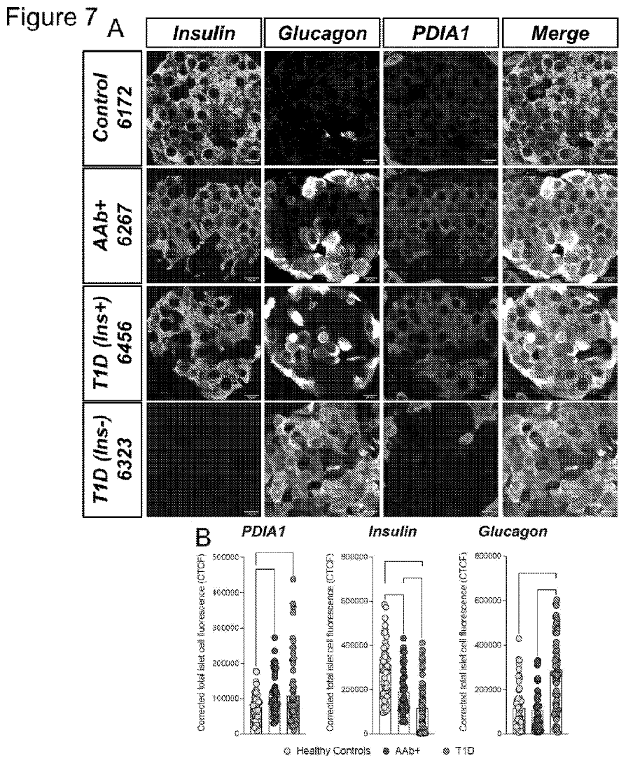

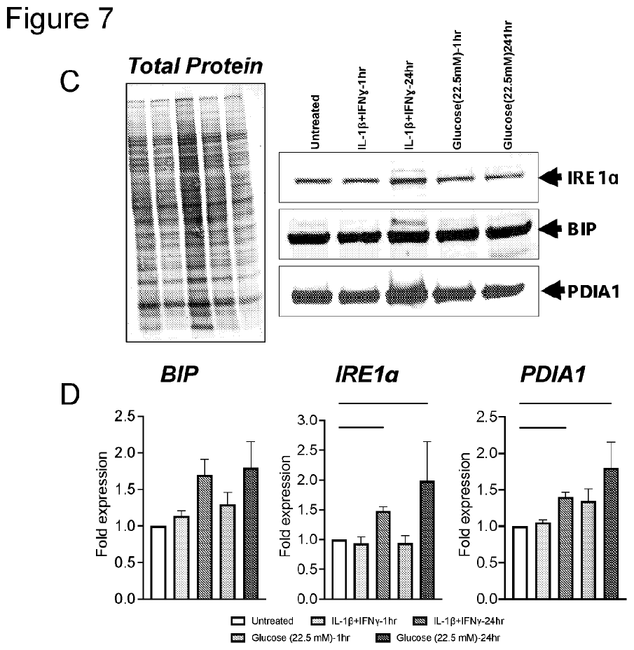

To test the relevance the findings in human subjects, pancreatic tissue sections were obtained from non-diabetic organ donors, organ donors with autoantibody positivity (AAb+), and organ donors with established T1D. Immunofluorescence analysis of PDIA1, insulin, and glucagon was performed and revealed a significant increase in PDIA1 expression in pancreatic islets of individuals with AAb+ and with TID compared to non-diabetic control donors (FIG. 7A-7B).

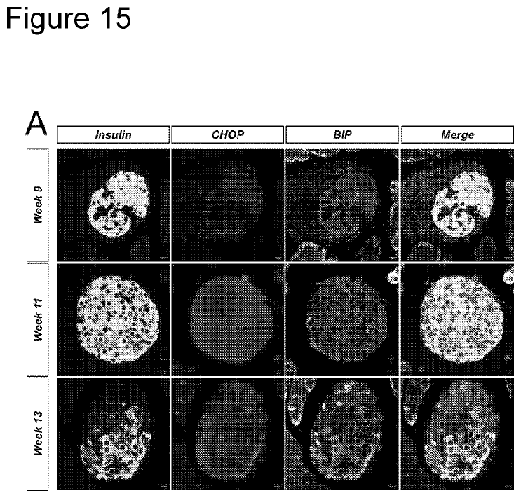

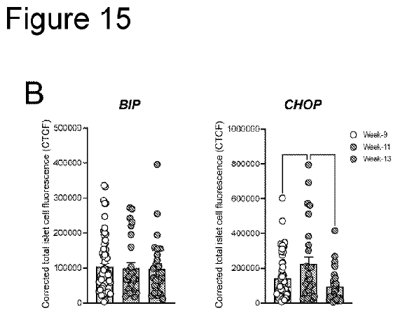

PDIA1 is an ER resident protein with an established role in proinsulin maturation. Therefore, to understand whether there was an association between ER stress and PDIA1 under conditions of β cell stress, an in vitro approach was undertaken. The in vitro approach comprised of treating human islets with or without pro-inflammatory cytokines (IL-1β+IFNg) or high glucose (22.5 mM) for 1 hour and 24 hours. Under both chronic stress conditions (i.e. 24 hour treatment), a parallel upregulation of PDIA1 and IRE1α was observed (FIG. 7C-7D). BIP expression was increased but not to a significant extent. In line with this data, immunofluorescence analysis of NOD pancreatic tissue sections showed a significant upregulation of CHOP at week 11 (FIG. 15A-B). However, there was no difference in BIP expression between different age groups of NOD mice.

### Example 19: Analysis of Circulating PDIA1 as a TID Associated Biomarker

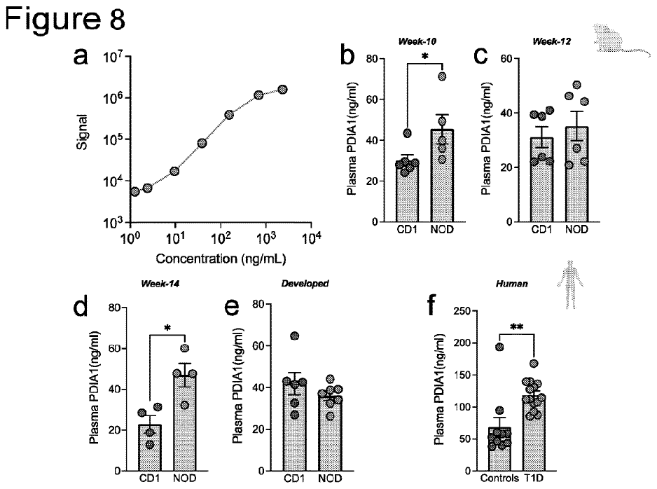

In addition to its intracellular role as a thiol reductase, PDIA1 is known to be a secreted protein. At present, circulating biomarkers that reflect the health of the β cell are lacking. Therefore, to determine whether the islet-specific upregulation of PDIA1 identified in the proteomics and immunofluorescence analyses was linked with changes in circulating levels of PDIA1, a high-sensitivity electrochemiluminescence assay using Meso Scale Discovery technology was developed. PDIA1 was measured using serially diluted (1:4) recombinant PDIA1, and this analysis showed that PDIA1 could be detected in the range of 0.152 ng/ml up to 2500 ng/ml (FIG. 8A). Using plasma collected from the same mice employed in the longitudinal proteomics analysis shown in FIG. 2, it was found that the plasma levels of PDIA1 were significantly increased in pre-diabetic NOD mice compared to CD1 mice at 10 and 14 weeks of age (FIGS. 8B-8D). However, PDIA1 levels were not different between NOD mice at the time of diabetes onset and age-matched CD1 mice (FIG. 8E). PDIA1 levels were below the detectable range in plasma from diabetic NOD-SCID mice that had undergone T-cell adoptive transfer.

Next, this assay was applied to serum samples collected from children within 48 hours of the clinical onset of T1D (n=10; average age=11.57±4.05 years; 8 male; 6 female) and in serum collected from non-diabetic pediatric controls (n=10; average age=12.1±4.20; 6 male; 4 female). (Table 1). Interestingly, serum levels of PDIA1 were significantly higher in pediatric subjects with recent-onset T1D compared to controls, suggesting PDIA1 may have utility as a clinical, human TID biomarker. (FIG. 8F). The PDIA1 levels in pediatric subjects may be about 10% to about 70% more than PDIA1 levels in control, including any percentage of range comprised therein. For example, the PDIA1 levels in pediatric subjects may be about 10% to about 20% more, from about 20% to about 40% more, or from about 40% to about 70% more than PDIA1 levels in control.

TABLE 1
Clinical and Anthropometric Characteristics

| | | Sex | | | *Blood Pressure (mmHg) | |
|---|---|---|---|---|---|---|
| | *Age (years) | (M/F) | *BMI % | *HbA1c | Systolic | Diastolic |
| Healthy | 12.1 ± 4.2 yrs | 6M/4F | 66.3 ± 24.33 | N/A | 108 ± 11.35 | 63 ± 9.3 |
| Controls | | | | | | |
| New Onset | 11.57 ± 4.05 yrs | 8M/6F | 55.42 ± 28.37 | 12.84 ± 1.67% | 102 ± 11.2 | 57 ± 6.88 |
| T1D | | | | | | |

*Mean ± S.D.

1. A method of identifying a biomarker associated with early onset type 1 diabetes in humans, said method comprising:
performing a first analysis of a proteome of islets from female NOD mice at three pre-diabetic time points and at a time of diabetes onset;
performing a second analysis of proteome of islets from diabetic NOD mice and NOD-SCID mice that has been rendered acutely diabetic following an adoptive transfer of T cells from NOD.BDC2.5 mice;
performing a third analysis of islets from NOD mice that remained diabetes-free after a time period of observation; and
identifying a murine biomarker based on the first, second, and third analysis; and
using the murine biomarker as the biomarker associated with early onset type 1 diabetes in humans before administering a type 1 diabetes preventative therapeutic.

2. The method of claim 1, wherein the time period of observation ranges from about 46 weeks to about 48 weeks.

3. The method of claim 1, wherein the murine biomarker is protein disulfide isomerase A1 (PDIA1) or protein 14-3-3b.

4. A method for treating subjects at risk for developing type 1 diabetes, the method comprising:
identifying a subject at risk of type 1 diabetes by measuring protein disulfide isomerase A1 (PDIA1) levels in a body fluid of the subject;
measuring protein disulfide isomerase A1 (PDIA1) levels in a body fluid of a control;
comparing the relative concentration of PDIA1 levels in the body fluid of the subject to PDIA1 levels in the body fluid of the control;
determining that the subject has type 1 diabetes when the concentration of PDIA1 levels in the body fluid of the subject is statistically greater than the concentration of PDIA1 levels in the body fluid of the control; and
administering a type 1 diabetes preventative therapeutic if the subject has type 1 diabetes.

5. The method claim 4, wherein the type 1 diabetes preventative therapeutic comprises restricting carbohydrate consumption by the subject.

6. The method claim 4, wherein the type 1 diabetes preventative therapeutic comprises increasing fluid consumption.

7. The method claim 4, wherein the type 1 diabetes preventative therapeutic comprises administering an immunotherapy agent, optionally wherein the immunotherapy agent is teplizumab.

8. The method claim 4, wherein the body fluid of the subject is blood or serum.

9. The method of claim 5 wherein the type 1 diabetes preventative therapeutic comprises a compound selected from the group consisting of a nonsteroidal anti-inflammatory drug, a corticosteroid, and an immune-suppressant drug.

10. The method of claim 4, further comprising:
measuring protein 14-3-3b levels in the body fluid of the subject;
measuring protein 14-3-3b levels in the body fluid of the control;
comparing the relative concentration of 14-3-3b levels in the body fluid of the subject to 14-3-3b levels in the body fluid of the control;
determining that the subject has type 1 diabetes when the concentration of PDIA1 and 14-3-3b levels in the body fluid of the subject are statistically greater than the concentration of PDIA1 levels and 14-3-3b levels in the body fluid of the control; and
administering a type 1 diabetes preventative therapeutic if the subject has type 1 diabetes.

11. A method for treating subjects at risk for developing type 1 diabetes, the method comprising:
identifying a subject at risk of type 1 diabetes by
measuring a first biomarker level in a body fluid of the subject;
measuring the first biomarker level in a body fluid of a control;
comparing the relative concentration of the first biomarker in the body fluid of the subject to first biomarker level in the body fluid of the control;
determining that the subject has type 1 diabetes when the concentration of the first biomarker level in the body fluid of the subject is statistically greater than the concentration of the first biomarker level in the body fluid of the control; and
administering a type 1 diabetes preventative therapeutic if the subject has type 1 diabetes;
wherein the first biomarker is associated with maintaining β cell function.

12. The method claim 11, wherein the subject is a pediatric subject, and wherein the first biomarker level in the subject was measured within 48 hours of the clinical onset of type 1 diabetes.

13. The method claim 11, wherein the first biomarker level in the body fluid of the subject is about 10% to about 70% more than the first biomarker level in the second body fluid of the control.

14. The method claim 11, wherein the body fluid of the subject is serum.

15. The method claim 11, wherein the first biomarker is PDIA1.

16. The method claim 11, wherein the method further comprises measuring and comparing the relative concentration of a second biomarker in the body fluid of the subject to a second biomarker level in the second body fluid of the control before determining that the subject has type 1 diabetes and administering the type 1 diabetes preventative therapeutic.

17. (canceled)

18. The method claim 11, wherein the first biomarker is identified by methods comprising:
performing a first analysis of a proteome of tissue from female NOD mice at three pre-disease time points and at a time of disease onset;
performing a second analysis of proteome of tissue from diseased NOD mice and NOD-SCID mice that has been rendered to have the disease following an adoptive transfer of T cells from NOD.BDC2.5 mice;
performing a third analysis of tissue from NOD mice that remained disease-free after a time period of observations; and
identifying the first biomarker based on the first, second, and third analysis.

19. The method claim 18, wherein the disease is type 1 diabetes.

20. The method claim 18, wherein the first biomarker is associated with maintaining β cell function.

21. The method claim 19, wherein the first biomarker is protein disulfide isomerase A1 (PDIA1) or protein 14-3-3b.
